(19) United States
(12) Patent Application Publication
(10) Pub. No.: US 2017/0312201 A1
(43) Pub. Date: Nov. 2, 2017

(54) PHOTOCURABLE COMPOSITION FOR TOPCOAT OF NAILS OR ARTIFICIAL NAILS

(71) Applicant: THREE BOND CO., LTD., Hachioji-shi, Tokyo (JP)

(72) Inventor: Koichi TAKEMOTO, Hachioji-shi, Tokyo (JP)

(21) Appl. No.: 15/523,786

(22) PCT Filed: Oct. 29, 2015
(86) PCT No.: PCT/JP2015/080611

§ 371 Date: May 2, 2017

(30) Foreign Application Priority Data
Nov. 5, 2014 (JP) 2014-224794

(51) Int. Cl. A61K 8/37; A61K 8/362; A61K 8/35; A61K 8/55; A61Q 3/02; A61K 8/87

(52) U.S. Cl. CPC A61K 8/37; A61Q 3/02; A61K 8/87; A61K 8/35; A61K 8/55; A61K 8/362; A61K 2800/81

(57) ABSTRACT

The object of the present invention is to provide a photocurable composition for a topcoat of nails or artificial nails having excellent glossiness and a colorless and transparent outer appearance.

The present invention is related to a photocurable composition for a topcoat of nails or artificial nails including the following components (A) to (C), including 40 to 70% by mass of trimethylol propane trimethacrylate relative to the total amount of the component (B), and satisfying that a cured product thereof with a thickness of 800 μm has transmittance of 10.0% or higher at a wavelength of 400 nm:

component (A); a (meth)acrylic oligomer;

component (B): a trifunctional (meth)acrylic monomer contained at 50 to 100 parts by mass relative to 100 parts by mass of the component (A); and

component (C): a photopolymerization initiator contained at 1 to 20 parts by mass relative to 100 parts by mass of the component (A).

### TECHNICAL FIELD

The present invention relates to a photocurable composition for a topcoat of nails or artificial nails. In particular, the present invention relates to a photocurable composition which is used for a topcoat to exhibit glossiness on a surface of nails or artificial nails and to protect an under layer.

### BACKGROUND ART

A photocurable composition used for a topcoat of nails or artificial nails is known in a related art, and it is described in JP 2013-506667 B (corresponding to the specification of US 2011/082,228 A), for example. It is described in the literature that, since the topcoat formed of this composition does not require any polishing treatment for removal process, a topcoat can be rapidly removed, that is, removal can be achieved in less than 20 minutes, compared to a photocurable topcoat of a related art which uses a solvent.

### SUMMARY OF INVENTION

However, an easily removable topcoat has poor curing on a surface inevitably so that it cannot play the role of a topcoat. It is also considered that, to have a beautifully looking decoration in a base, a transparent and colorless outer appearance are most important for a topcoat as well as glossiness. However, since an acrylic compound or a methacrylic compound used in a photocurable composition causes an oxygen inhibition, the outermost surface of a topcoat which is in contact with air is difficult to be cured resulting in residual stickiness and a poor outer appearance. For such reasons, in the field of gel nails, after curing the topcoat by light irradiation, an operation of wiping out a non-cured part on the outermost surface with a piece of cloth impregnated with a solvent is carried out.

With regard to a photocurable composition which is used for a topcoat of nails or artificial nails, if an operation of wiping out a non-cured part of a topcoat with a solvent is carried out under the premise that oxygen inhibition occurs after curing, it is difficult to maintain the glossiness and a colorless and transparent outer appearance of a topcoat.

Under the circumstances, an object of the present invention is to provide a photocurable composition for a topcoat of nails or artificial nails which enables exhibition of excellent glossiness and a colorless and transparent outer appearance even when the aforementioned wiping operation is carried out after curing.

To solve the problems that are described above, the inventors of the present invention conducted intensive studies, and as a result, found that the problems can be solved by a photocurable composition for a topcoat nails or artificial nails having following constitutions. Accordingly, the present invention is completed.

1. A photocurable composition for a topcoat of nails or artificial nails including the following components (A) to (C), including 40 to 70% by mass of trimethylol propane trimethacrylate relative to the total amount of the component (B), and satisfying that a cured product thereof with a thickness of 800 μm has transmittance of 10.0% or higher at a wavelength of 400 nm:

component (A): a (math) acrylic oligomer;

component (B): a trifunctional (meth)acrylic monomer contained at 50 to 100 parts by mass relative to 100 parts by mass of the component (A); and

component (C): a photopolymerization initiator contained at 1 to 20 parte by mass relative to 100 parts by mass of the component (A).

2. A photocurable composition for a topcoat of nails or artificial nails including the following components (A) to (C), including 40 to 70% by mass of trimethylol propane trimethacrylate relative to the total amount of the component (B), and including 0 to 30% by mass of a visible light type photopolymerization initiator relative to the total amount of the component (C):

component (A): a (meth) acrylic oligomer;

component (B): a trifunctional (meth)acrylic monomer contained at 50 to 100 parts by mass relative to 100 parts by mass of the component (A); and

component (C): a photopolymerization initiator contained at 1 to 20 parts by mass relative to 100 parts by mass of the component (A).

3. The photocurable composition for a topcoat of nails or artificial nails according to 2 described above, wherein a cured product thereof with a thickness of 800 μm has transmittance of 10.0% or higher at a wavelength of 400 nm.

4. The photocurable composition for a topcoat of nails or artificial nails according to any one of 1 to 3 described above, further including a plasticizing agent as the component (D).

5. A coating film obtained by curing the photocurable composition for a topcoat of nails or artificial nails according to any one of 1 to 4 described above by light irradiation and then wiping out a surface of the cured product with a piece of cloth impregnated with a solvent.

6. A method for exhibiting glossiness by curing the photocurable composition for a topcoat of nails or artificial nails according to any one of 1 to 4 described above by light irradiation and then wiping out a surface of the cured product with a piece of cloth impregnated with a solvent.

### DESCRIPTION OF EMBODIMENTS

One embodiment of the present invention is a photocurable composition for a topcoat of nails or artificial nails containing component (A): a (meth) acrylic oligomer, component (B): a trifunctional (meth)acrylic monomer included at 50 to 100 parts by mass relative to 100 parts by mass of the component (A), and component (C): a photopolymerization initiator included at 1 to 20 parts by mass relative to 100 parts by mass of the component (A), containing trimethylol propane trimethacrylate at 40 to 70% by mass relative to the total amount of the component (B), and satisfying at least one of the following (i) and (ii):

(i) a cured product thereof with a thickness of 800 μm has transmittance of 10.0% or higher at a wavelength of 400 nm; and

(ii)a visible light type photopolymerization initiator is included at 0 to 30% by mass relative to the total amount of the component (C).

Since the photocurable composition for a topcoat of nails or artificial nails with the above constitution has low viscosity and exhibits low heat generation upon curing by light irradiation with ultraviolet ray or visible light after coating on nails. Therefore, the composition has excellent workability. Furthermore, a coating film obtained by curing of the composition can exhibit excellent glossiness and a colorless and transparent outer appearance even when an operation of wiping out a non-cured part on the outermost surface with a solvent is carried out.

Hereinbelow, the photocurable composition for a topcoat of nails or artificial nails according to the present invention (hereinbelow, referred to as a “photocurable composition”, or simply as a “composition”) and a cured product formed of the composition will be described in detail. Meanwhile, in the present specification, the description “X to Y” is used as a meaning in which the numerical values that are described before and after the description (that is, X and Y) are included as a lower limit and an upper limit, respectively. Furthermore, unless specifically described otherwise, operations and measurements of physical properties or the like are carried out at conditions including room temperature (25° C.)/relative humidity of 40 to 50%.

### Photocurable Composition

Hereinbelow, each constitutional, component of the composition of the present invention will be described.

### Component (A)

In the composition of the present, invention, a (math) acrylic oligomer having an acrylic group and/or a methacrylic group is contained as the component (A). Hereinbelow, both the acrylic group and methacrylic group are referred to as a (meth) acrylic group. The binding position of a (meth)acrylic group is not particularly limited, and it may be present at an end or in a main skeleton of a molecule. As the component (A), it is preferable to use a compound which is in a liquid phase under an atmosphere of 25° C. and has good compatibility with the components (B) to (D) that are described below.

Specific examples of the (meth) acrylic oligomer include a (meth) acrylic oligomer with an ester bond, a (meth) acrylic oligomer with an ether bond, a (meth) acrylic oligomer with a urethane bond, and an epoxy modified (meth) acrylic oligomer, and examples of the main skeleton thereof include bisphenol A, novoloc phenol, polybutadiene, polyester, and polyether, but not limited thereto. Furthermore, as the component (A) of the present invention, a compound which has at least one epoxy group and at least one (meth) acrylic group in one molecule can be also used.

With regard to the (meth) acrylic oligomer with an ester bond, known is a synthesis in which an ester bond is formed based on synthesis of polyol and polyhydric carboxylic acid and then acrylic acid is added to a non-reacted hydroxyl group, but it is not limited to this synthetic method. Specific examples thereof include. ARONIX M-6100, M-6200, M-6250, M-6500, M-7100, M-7300K, M-8030, M-8060, M-8100, M-8530, M-8560, M-9050, arid the like that are manufactured by TOAGOSEI CO., LTD., and UV-3500BA, UV-3520TL, UV-3200B, UV-3000B and the like that are manufactured by Nippon Synthetic Chemical Industry Co., Ltd., but not limited thereto.

With regard to the (meth)acrylic oligomer with an ether bond, known is a synthetic method in which acrylic acid is added, to a hydroxyl group of polyether polyol or an aromatic hydroxyl group of bisphenol or the like, but it is not limited to this synthetic method. Specific examples thereof include UV-6640B, UV-6100B, UV-3700B, and the like that, are manufactured by Nippon Synthetic Chemical Industry Co., Ltd., LIGHT (math) acrylate 3EG-A, 4EG-A, 9EG-A, 14EG-A, PTMGA-250, BP-4EA, BP-4PA, BP-10EA, and the like that are manufactured by KYOEISHA CHEMICAL Co., Ltd., and EBECRYL3700 and the like that are manufactured by Daicel-Cytec Co., Ltd., but not limited thereto.

With regard to the (meth) acrylic oligomer with a urathane bond, known is an oligomer which is synthesized by forming a urethane bond using polyol and polyisocyanate and then adding acrylic acid to a non-reacted hydroxyl group. Specific examples thereof include AH-600, AT-600, UA-306H, UF-8001G, and the like that are manufactured by KYOEISHA CHEMICAL Co., Ltd., but not limited thereto. Incidentally, the component (A) may be used either singly or in combination, of two or more kinds thereof.

### Component (B)

In the composition of the present invention, a trifunctional (meth) acrylic monomer is contained as the component (B). Specific examples thereof include trimethylol propane tri(meth)acrylate, ethoxylated trimethylol propane (meth) acrylate, isocyanuric acid EO modified tri (meth)acrylate, pentaerythritol tri(meth)acrylate, EO modified trimethylol propane tri(meth)acrylate, PO modified trimethylol propane tri(meth)acrylate, ECH modified trimethylol propane tri (meth) acrylate, ECH modified glycerol tri (meth) acrylate, and tris(acryloyloxyethyl)isocyanurate. Among them, trimethylol propane tri (meth) acrylate, ethoxylated trimethylol propane tri (meth) acrylate, and isocyanuric acid EO modified tri (meth) acrylate are preferable. The component (B) may be used either singly or in combination of two or more kinds thereof. However, from the viewpoint of the glossiness of a cured product which is formed of the composition, trimethylol propane trimethacrylate is essentially contained (see Example 3 and Comparative Example 5 that are shown below).

In the present invention, content of the component (B) is 50 to 100 parts by mass, and more preferably 53 to 95 parts by mass, relative to 100 parte by mass of the component (A). From the viewpoint of the hardness of a cured product formed, of the composition, it is even more preferably 57 to 90 parts by mass relative to 100 parts by mass of the component (A). When the component (B) is present at 50 mass or more, the photocurability of the composition can be maintained. When (B) is present, at 100 parts by mass or less, the composition can maintain the storage stability without being thickened during storage. On the other hand, when the component (B) is less than 50 parts by mass, glossiness is not exhibited after wiping out a surface of a cured product formed of the composition, with a piece of cloth (see Comparative Example 11 that is shown below).

Furthermore, in the present invention, content of trimethylol propane trimethacrylate is 40 to 70% by mass relative to the total amount of the component (B). With such content, glossiness can be exhibited on a surface of a cured product by, after curing of the composition, wiping out with a piece of cloth impregnated with a solvent. Furthermore, from the viewpoint of the hardness of a cured product formed of the composition, content of trimethylol propane trimethacrylate is preferably 40 to 63% by mass, and more preferably 45 to 63% by mass, relative to the total amount of the component (B). On the other hand, when the content of trimethylol propane trimethacrylate is less than 40% by mass or more than 70% by mass relative to the total amount of the component (B), no glossiness is exhibited after wiping out a surface of a cured product formed of the composition with a piece of cloth (for a case in which the content is lower than the lower limit, see Comparative Examples 7, 10, and 12 that are shown, below, and for a case in which the content is higher than the upper limit, see Comparative Example 13 that is shown below).

It is also preferable to use, as the component (B) of the present invention, isocyanuric acid EO modified, tri(meth)acrylate. Accordingly, the hardness of a cured product formed of the composition can be enhanced. Content of the isocyanuric acid EO modified tri(meth)acrylate is preferably 0 to 60% by mass, more preferably 15 to 50% by mass, even more preferably 25 to 50% by mass, and particularly preferably 40 to 45% by mass, relative to the total amount of the component (B). With such content, the hardness of a cured product formed of the composition can be further enhanced.

### Component (C)

In the composition of the present invention, a photopolymerization initiator is contained as the component (C).

The component (C) is not particularly limited as long as it is a radical-based photopolymerization initiator which can generate radical species by an energy ray such as visible, light, ultraviolet ray, X ray, or electron beam. Specific examples of the radical-based photopolymerization initiator include acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl) ketone, 1-hydroxycyclohexyl phenyl ketone, 2-hydroxy-1-{4-[4-(2-hydroxy-2-methyl-propionyl)-benzyl]phenyl}-2-methyl-propan-1-one, methylbenzoyl formate, 2-methyl-2-morpholino(4-thiomethylphenyl)propan-1-one, 2-benzyl-2-dimethylamino-1-(4-morpholinophenyl) butanone, and

2-hydroxy-2-methyl-1-[4 -(1-methylvinyl) phenyl]propanone oligomer; benzoins such as benzoin, benzoin methyl ether, benzoin ethyl ether, benzoin isopropyl ether, or benzoin isobutyl ether; benzophenones such as benzophenone, methyl o-benzoyl benzoate, 4-phenylbenzophenone, 4-benzoyl-4′-methyl-diphenyl sulfide, 3,3′, 4,4′-tetra(t-butylperoxycarbonyl)benzophenone, 2,4,6-trimethylbenzophenone,

4-benzoyl-N,N-dimethyl-N-[2-(1-oxo-2-propenyloxy) ethyl]benzenemethane aminum bromide, or (4-benzoyl benzyl) trimethylammonium chloride; and thioxanthones such as 2-isopropylthioxanthone, 4-isopropylthioxanthone, 2,4-diethylthioxanthone, 2,4-dichlorothioxanthone, 1-chloro-4-propoxythioxanthone, or 2(3-dimethylamino-2-hydroxy)-3,4-dimethyl-9H-thioxantho n-9-one mesochloride. Among them, from the viewpoint of a universal application property, 1-hydroxycyclohexyl phenyl ketone,

2-hydroxy-1-{4-[4-(2-hydroxy-2-methyl-propionyl)-benzyl]phenyl}-2-methyl-propan-1-one, methylbenzoyl formate, and 2-hydroxy-2-methyl-1-phenylpropan-1-one are preferable, and 1-hydroxycyclohexyl phenyl ketone is particularly preferable. The radical-based photopolymerization initiator may be used either singly or in combination of two or more kinds thereof.

As for the component (C) of the present invention, it is also possible to use a cationic photopolymerization initiator instead of the above radical-based photopolymerization initiator or a cationic photopolymerization initiator in combination of the above radical-based photopolymerization initiator. Specific examples of the cationic photopolymerization initiator include a diazonium salt, a sulfonic salt, and an iodonium salt. Specifically, there are benzene diazonium. hexafluoroantimonate, benzene diazonium hexafluoro phosphate, benzene diazonium hexafluoroborate, triphenylsulfonium hexafluoroantimonate, triphenylsulfonium hexafluorophosphate, triphenylsulfonium hexafluoroborate,

4,4′-bis [bis (2-hydroxyethoxyphenyl) sulfonic]phenyl sulfide bishexafluorophosphate, diphenyliodonium hexafluoroantimonate, diphenyliodonium hexafluorophosphate, and diphenyl-4-thiophenoxyphenylsulfonium hexafluoro phosphate, but not limited thereto. The cationic photopolymerization initiator may be used either singly or in combination of two or more kinds thereof.

Furthermore, from the viewpoint of inhibiting yellowing at the time of curing of the composition, it is preferable that a visible light type photopolymerization initiator is included as the component (C). The visible light type photopolymerization initiator is a photopolymerization initiator which exhibits the maximum light absorption in a visible light range, and it mainly indicates an acylphosphine oxide-based photopolymerization initiator having a phosphorus atom. Specific examples of the visible light type photopolymerization initiator include 2,4,6- trimethylbenzoyl-diphenyl-phosphine oxide and bis(2,4,6-trimethylbenzoyl)-phenylphosphine oxide. Among them, from the viewpoint of enhancing the photocurability, 2,4,6-trimethylbenzoyl-diphenyl-phosphine oxide is particularly preferable.

In the present invention, content of the component (C) is 1 to 20 parts by mass relative to 100 parts by mass of the component (A). When the component (C) is present at 1 part by mass or more, the photocurability of the composition can be maintained. When the component (C) is present at 20 parts by mass or less, the composition can maintain the storage stability without being thickened during storage.

Furthermore, when the aforementioned visible light type photopolymerization initiator is contained as the component (C), content thereof is preferably 0 to 30% by mass, and more preferably 10 to 20% by mass relative to the total amount of the component (C). With such content, yellowing does not occur at the time of curing of the composition. On the other hand, when the content of the visible light type photopolymerization initiator is more than 30% by mass relative to the entire amount of the component (C), glossiness is not exhibited after wiping out a surface of a cured product formed of the composition with a piece of cloth and also coloration of the cured product is observed, resulting in a damage in the outer appearance of the cured product (see Comparative Examples 6 and 7 that are shown below).

### Component (D)

The composition of the present invention preferably contains, as the component (D), a plasticizing agent other than the components (A) to (C). Specific examples of the plasticizing agent include aromatic polycarboxylic acid ester as a polycarboxylic acid ester-based plasticizing agent, dioctyl phthalate (DOP), dibutyl phthalate (DBP), diheptyl phthalate (DHP), diisononyl phthalate (DINP), diisodecyl phthalate (DIDP), and butylbenzy phthalate (BBP) as phthalic acid ester, trioctyl trimellitic acid (TOTM) and triisodecyl trimellitic acid (TITM) as trimellitic acid ester, tetraoctyl pyromellitic acid as pyromellitic acid ester, and di 2-ethylhexyl adipic acid (DOA), isodecyl adipic acid (DIDA), di 2-ethylhexyl sebacic acid (DOS), dibutyl sebacic acid (DBS), di 2-ethylhexyl maleic acid (DOM), dibutyl furmaric acid (DBF), di 2-ethylhexyl azellaic acid (DOZ), di 2-ethylhexyl epoxyhexahydrophthalic acid, trioctyl citric acid, and glycerol triacetate as aliphatic polycarboxylic acid ester, but not limited thereto. Furthermore, examples of the phosphoric acid-based plasticizing agent include trimethyl phosphate, tributyl phosphate, tri-(2-ethylhexyl) phosphate, tributoxyethyl phosphate, triphenyl phosphate, tricresyl phosphate, alkyl aryl phosphate, triethyl phosphate, tris(chloroethyl) phosphate, trisdichloropropyl phosphate, tris(β-chloropropyl) phosphate, octyl diphenyl phosphate, tris (isopropylphenyl) phosphate, and cresyl phenyl phosphate. Among them, from the viewpoint of a universal application property, di 2-ethylhexyl sebacic acid (DOS) is preferable. The plasticizing agent initiator may be used either singly or in combination of two or more kinds thereof.

According to the present, invention, content of the component (D) is preferably 0.1 to 10 parts by mass relative to 100 parts by mass of the component (A). When the component (D) is present at 0.1 parts by mass or more, it is difficult to have a scratch at the time of wiping out a surface of a cured product formed, of the composition with a piece of cloth impregnated with a solvent, and also exhibition of glossiness is obtained. When the component (D) is present at 10 parts by mass or less, it is unlikely for the cured product formed of the composition to have a scratch over time, and the glossiness can be maintained. Accordingly, a preferred embodiment of the present invention is a photocurable composition for a topcoat of nails or artificial nails further containing a plasticizing agent as the component (D).

### Other Monomers

In the composition of the present invention, other monomers may be contained in addition to the components (A) to (D). Other monomers are not particularly limited and preferably contain a monofunctional and/or bifunctional (meth)acrylic monomer from the viewpoint of having copolymerization with the component (A) and the component (B).

Specific examples of the monofunctional (meth)acrylic monomer include (meth)acrylic acid, lauryl (meth) acrylate, stearyl (meth)acrylate, ethylcarbitol (meth)acrylate, tetrahydrofurfuryl (meth)acrylate, caprolactone modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, phenoxyethyl (meth)acrylate, phenoxydiethylene glycol (meth)acrylate, phenoxytetraethylene glycol (meth)acrylate, nonylphenoxyethyl (meth) acrylate, nonylphenoxytetraethylene glycol (meth)acrylate, methoxydiethylene glycol (meth)acrylate, ethoxydiethylene glycol (meth) acrylate, butoxyethyl (meth) acrylate, butoxytriethylene glycol (meth)acrylate, 2-ethylhexylpolyethylene glycol (meth)acrylate, nonylphenylpolypropylene glycol (meth)acrylate, methoxy dipropylene glycol (meth) acrylate, glycidyl (meth) acrylate, 2-hydroxyethyl (meth)acrylate, 2-hydroxypropyl (meth)acrylate, glycerol (meth)acrylate, polyethylene glycol (meth)acrylate, polypropylene glycol (meth)acrylate, epichlorohydrin (ECH) modified butyl (meth)acrylate, ECH modified phenoxy (meth) acrylate, ethylene oxide (EO) modified phthalic acid (meth) acrylate, EO modified succinic acid (meth)acrylate, caprolactone modified 2-hydroxyethyl (meth)acrylate, N,N-dimethylaminoethyl (meth)acrylate, N,N-diethylaminoethyl (meth)acrylate, morpholino(meth)acrylate, and EO modified phosphoric acid (meth)acrylate. Among them, from the viewpoint of enhancing the hardness of a cured product, a (meth) acrylic monomer having an alicyclic structure is preferable, and isobornyl (meth) acrylate is particularly preferable. Alternatively, a monofunctional monomer having a hydroxyl group is preferable. Specific examples thereof include 2-hydroxyethyl (meth)acrylate and 2-hydroxypropyl (meth)acrylate, but not limited thereto. Those compounds may be used either singly or in combination of two or more kinds thereof.

The alicyclic structure represents a cyclic structure of hydrocarbons having no unsaturated, bond, and specific examples thereof include a cycloalkyl group such as a cyclopropyl group, a cyclobutyl group, a cyclopentyl group, a cyclohexyl group, a butylcyclohexyl group, a methylcyclohexyl group, a dimethyleyelohexyl group, a cycloheptyl group, a methylcycloheptyl group, a cyclooctyl group, a cyclononyl group, or a cyclodecyl group, a hydronaphthyl group, a 1-adamantyl group, a 2-adamantyl group, a norbornyl group, a methylnorbornyl group, an isobornyl group, a dicyclopentenyl group, a dicyclopentanyl group, and a dicyclopentenyloxyethyl group.

Content of the mono functional (meth) acrylic monomer is, although not particularly limited, preferably 0 to 50 parts by mass relative to 100 parts by mass of the component (A). Furthermore, from the viewpoint of the workability, it is more preferably 10 to 45 parts by mass s and even more preferably 25 to 40 parts by mass.

Specific examples of the bifunctional (meth)acrylic monomer include isocyanuric acid EO modified di (meth) acrylate, 1,3-butylene glycol di(meth)acrylate, 1,4-butylene glycol di(meth)acrylate, dimethylol tricyclodecane di(meth)acrylate, neopentyl glycol di(meth)acrylate, 1,6-hexane glycol di(meth)acrylate, ethylene glycol diacrylate, polyethylene glycol di (meth) acrylate, propylene glycol di(meth)acrylate, tripropylene glycol di(meth)acrylate, EO modified neopentyl glycol di(meth)acrylate, propylene oxide (PO) modified neopentyl glycol di(meth)acrylate, bisphenol A di(meth)acrylate, EO modified bisphenol A di (meth) acrylate, ECH modified bisphenol A di (meth) acrylate, EO modified bisphenol S di (meth) acrylate, hydroxypyvalic acid ester neopentyl glycol di (meth) acrylate, caprolactone modified hydroxypyvalic acid ester neopentyl glycol di(meth)acrylate, neopentyl glycol modified trimethylol propane di (meth) acrylate, stearic acid, modified pentaerythritol di(meth)acrylate, dicyclopentenyl di(meth)acrylate, EO modified dicyclopentenyl di (meth) acrylate, and di (meth) acryloyl isocyanurate. Among them, from the viewpoint of enhancing the hardness of a cured product, a bifunctional (meth)acrylic monomer having an alicyclic structure is preferable, and isocyanuric acid EO modified di(meth)acrylate and dimethylol tricyclodecane di (meth) acrylate are particularly preferable. Furthermore, those compounds may be used either singly or in combination of two or more kinds thereof.

Content of the bifunctional (meth)acrylic monomer is, although not particularly limited, preferably 2 to 30 parts by mass relative to 100 parts by mass of the component (A). Furthermore, from the viewpoint of the hardness of a cured product which is formed of the composition, it is more preferably 10 to 25 parts by mass, and even more preferably 15 to 20 parts by mass.

According to the present invention, content of other monomers is preferably 5 to 60 parts by mass, and more preferably 10 to 50 parts by mass, relative to 100 parts by mass of the component (A). When other monomers are present at 5 parts by mass or more, the photocurability of the composition can be maintained. When other monomers are present at 60 parts by mass or less, the storage stability of the composition can be maintained without being thickened during storage.

### Other Additives

Within a range in which the characteristics of the present invention are not adversely affected, it is possible for the composition of the present invention to be blended with a suitable amount of additives including a coloring agent such as a pigment and a dye, an inorganic filler such as metal powder, calcium carbonate, talc, silica, alumina, or aluminum hydroxide, a flame retardant, an organic filler, an anti-oxidant, a polymerization inhibitor, an anti-foaming agent, a coupling agent, a leveling agent, and a rheology controlling agent. According to addition of these additives, a composition with excellent resin strength, adhesion strength, workability, storage property, or the like, or a cured product of the composition can be obtained.

From the viewpoint of the workability, viscosity of the composition of the present invention is preferably 30 Pa·s or less at a temperature of 25° C. The viscosity indicates viscosity which is measured according to the method described in “Viscosity measurement” of the following examples. Furthermore, the composition of the present invention may be any one of a solvent-free type and a solvent type.

### Production of Cured Products

By applying the photocurable composition for a topcoat of the present invention on nails or artificial nails and then curing the photocurable composition by light irradiation, a cured product (that is, coating film) can be obtained. In that case, the light for irradiation may be any one of ultraviolet rays and visible light. The light source to be used is not particularly limited, and examples thereof which may be used include a low pressure mercury lamp, a medium pressure medium lamp, a high-pressure mercury lamp, an ultra-high pressure mercury lamp, a black light lamp, a microwave exited mercury lamp, a metal halide lamp, a sodium lamp, a halogen lamp, a xenon lamp, an LED, a fluorescent lamp, sun light, and a device for irradiating electron beam. Cumulative amount of light for light irradiation is preferably 30 kJ/m² or less from the viewpoint of an influence on skin. From the viewpoint of curing efficiency, it is preferably 5 kJ/cm² or more.

The cured product formed of the composition preferably has a colorless and transparent outer appearance without yellowing. Specifically, when a thickness of a cured product is 800 μm, the transmittance is preferably 10.0% or higher at a wavelength of 400 nm. Accordingly, a preferred mode of the present invention is a photocurable composition for a topcoat of nails or artificial nails, wherein a cured product thereof with a thickness of 800 μm has transmittance of 10.0% or higher at a wavelength of 400 nm. The “transmittance at a wavelength of 400 nm of a cured product, with a thickness of 800 μm” means the transmittance at a wavelength of 400 nm that is measured by the method described in [Measurement of transmittance] in the following examples.

The coating film obtained by curing of the composition of the present invention can exhibit, glossiness by wiping the non-cured part on a surface having oxygen inhibition using a piece of cloth impregnated with a solvent. The solvent to be used is not particularly limited as long as it is a solvent which can remove a non-cured monomer or oligomer on a surface of a cured product, and examples thereof include ethanol and isopropanol. Furthermore, as a piece of cloth to be used for wiping, cotton or the like can be mentioned, for example.

Accordingly, a preferred mode of the present invention is a coating film which is obtained by curing the photocurable composition for a topcoat of nails or artificial nails by light irradiation and then wiping out a surface of a cured product with a piece of cloth impregnated, with a solvent. The coating film obtained accordingly can exhibit glossiness even after the coating film surface is wiped out with s piece of cloth impregnated with a solvent. Accordingly, the present invention also provides a coating film which is obtained by curing the photocurable composition for a topcoat of nails or artificial nails of the present invention by light irradiation and then wiping out a surface of a cured product with a piece of cloth impregnated with a solvent to exhibit glossiness. As such, another preferred mode of the present invention is a method for exhibiting glossiness by curing the photocurable composition for a topcoat of nails or artificial nails by light irradiation and then wiping out a surface of a cured product with a piece of cloth impregnated with a solvent.

### EXAMPLES

Next, the present invention will be described in more details in view of Examples. However, the present invention is not limited to Examples. Incidentally, in the following Examples, the operations were performed at a room temperature (25° C.) unless specifically described otherwise. In addition, “%” and “parts” mean “% by mass” and “parts by mass”, respectively, unless specifically described otherwise.

### Examples 1 to 12 and Comparative Examples 1 to 13

The following components were prepared to produce a composition:

[Component (A): (Meth)Acrylic Oligomer]

urethane acryl oligomer (bifunctional, weight average molecular weight: 4500) (UF-8001G, manufactured by KYOEISHA CHEMICAL Co., LTD.) (in the following Table 1, referred to as “UF-8001G”)

[Component (B): Trifunctional (Meth)Acrylic Monomer]

trimethylol propane trimethacrylate (NK ester TMPT, manufactured by Shin Nakamura Chemical Co., Ltd.) (in the following Table 1, referred to as “TMPT”)

trimethylol propane triacrylate (NK ester A-TMPT, manufactured by Shin Nakamura Chemical Co., Ltd.) (in the following Table 1, referred to as “A-TMPT”)

ethoxylated (9) trimethylol propane triacrylate (Sartomer SR502, manufactured by Sartomer Company) (in the following Table 1, referred to as “SR502”)

isocyanuric acid EO modified di- and triacrylate (trifunctional: 65% by mass, bifunctional: 35% by mass) (Aronix M-313, manufactured by TOAGOSEI CO., LTD.) (in the following Table 1, the trifunctional component of the compound is referred, to as “M-313 (trifunctional)” and the bifunctional component of the compound is referred to as “M-313 (bifunctional)”, respectively)

### Other Monomers

dimethylol tricyclodecane diacrylate (Light acrylate DCP-A, manufactured by KYOEISHA CHEMICAL Co., LTD.) (in the following Table 1, referred to as “DCP-A”)

dipentaerythritolhexaacrylate (NK ester A-DPH, Shin Nakamura Chemical Co., Ltd.) (in the following Table 1, referred to as “A-DPH”)

isobornylmethacrylate (Light ester IB-X, manufactured by KYOEISHA CHEMICAL Co., LTD.) (in the following Table 1, referred to as “IB-X”)

isobornylacrylate (Light acrylate IB-XA, manufactured by KYOEISHA CHEMICAL Co., LTD.) (in the following Table 1, referred to as “IB-XA”)

[Component (C): Photopolymerization Initiator]

2,4,6-trimethylbenzoyl-diphenyl-phosphine oxide (visible light type photopolymerization initiator) (LUCIRIN TPO, manufactured, by BASF Japan Ltd.) (in the following Table 1, referred to as “TPO”)

1-hydroxycyclohexyl phenyl ketone (non-visible light type photopolymerization initiator) (Suncure 84, manufactured by Chemark Chemical Co., Ltd) (in the following Table 1, referred to as “84”)

2-hydroxy-1-{4-[4-{2-hydroxy-2-methyl-propionyl}-benzyl]phenyl}-2-methyl-propan-1-one (non-visible light type photopolymerization initiator) (IRGACURE 127, manufactured by BASF, Japan) (in the following Table 1, referred to as “127”).

methylbenzoyl formate (non-visible light type photopolymerization initiator) (Vicure 55, manufactured by Akso Nobel K.V.) (in the following Table 1, referred to as “55”)

2 -hydroxy-2-methyl-1-phenyl-propan-1-one (non-visible light type photopolymerization initiator) (DAROCUR 1173, manufactured by BASF, Japan) (in the following Table 1, referred to as “1173”)

[Component (D): Plasticizing Agent]

di 2-ethylhexyl sebacic acid (SANSO CIZER DOS, manufactured by New Japan Chemical Co., Ltd.) (in the following Table 1, referred to as “DOS”),

After weighing the component (A), the component (B), other monomers, and the component (D) in a stirring tank, stirring was carried out while degassing under vacuum for 30 minutes at a temperature of 25° C. After that, the component (C) was weighed, and stirring was carried out again while degassing under vacuum for 30 minutes at a temperature of 25° C. Detailed preparation amount is as shown in Table 1, and all the numerical values are expressed in parts by mass. Herein, since M-313includes a trifunctional component and a bifunctional component, they are described as “M-313 (trifunctional)” and “M-313 (bifunctional)”, respectively. Furthermore, “TMPT content ratio (%) relative to total of the component (B)” means the mass ratio of trimethylol propane trimethacrylate in the total of the component (B), and “TPO content ratio (%) relative to total of the component (C)” means the mass ratio of a visible light type photopolymerization initiator in the total of the component (C).

TABLE 1

| | Raw | Example | Example | Example | Example | Example | Example | Example | Example | Example |
|---|---|---|---|---|---|---|---|---|---|---|
| Component | materials | 1 | 2 | 3 | 4 | 5 | 6 | 7 | 8 | 9 |
| Component | UF-8001G | 100 | 100 | 100 | 100 | 100 | 100 | 100 | 100 | 100 |
| (A) | | | | | | | | | | |
| Component | TMPT | 26 | 35 | 35 | 35 | 35 | 35 | 35 | 35 | 35 |
| (B) | A-TMPT | | | | | | | | | |
| | SR502 | 9 | 9 | 9 | 9 | 9 | 9 | 9 | 9 | 9 |
| | M313 | 23 | 11 | 23 | 23 | 23 | 23 | 23 | 23 | 34 |
| | (Trifunctional) | | | | | | | | | |
| Other | M313 | 12 | 6 | 12 | 12 | 12 | 12 | 12 | 12 | 16 |
| monomers | (Bifunctional) | | | | | | | | | |
| | DCP-A | | | | | | | | | |
| | A-DPH | | | | | | | | | |
| | IB-X | | | | | | | | | |
| | IB-XA | 17 | 17 | 17 | 17 | 17 | 17 | 17 | 35 | 17 |
| Component | TPO | 2 | 2 | 2 | 2 | 2 | 2 | 2 | 2 | 2 |
| (C) | 84 | 8 | 8 | 8 | 8 | 8 | 8 | 13 | 8 | 8 |
| | 127 | | | | 4 | | | | | |
| | 55 | | | | | 4 | | | | |
| | 1173 | | | | | | 4 | | | |
| Component | DOS | 5 | 5 | 5 | 5 | 5 | 5 | 5 | 5 | 5 |
| (D) | | | | | | | | | | |

| | | | | | | | | | |
|---|---|---|---|---|---|---|---|---|---|
| Total | 202 | 193 | 211 | 215 | 215 | 215 | 216 | 229 | 228 |
| Total of the component (B) | 58 | 55 | 67 | 67 | 67 | 67 | 67 | 67 | 78 |
| TMPT content ratio (%) | 45 | 64 | 52 | 52 | 52 | 52 | 52 | 52 | 45 |
| relative to total of the | | | | | | | | | |
| component (B) | | | | | | | | | |
| Total of the component (C) | 10 | 10 | 10 | 14 | 14 | 14 | 15 | 10 | 10 |
| TPO content ratio (%) | 20 | 20 | 20 | 14 | 14 | 14 | 13 | 20 | 20 |
| relative to total of the | | | | | | | | | |
| component (C) | | | | | | | | | |

| | | | | | Com- | Com- | Com- | Com- | |
|---|---|---|---|---|---|---|---|---|---|
| | | | | | parative | parative | parative | parative | |
| | Raw | Example | Example | Example | Example | Example | Example | Example | Comparative |
| Component | materials | 10 | 11 | 12 | 1 | 12 | 3 | 4 | Example 5 |
| Component | UF-8001G | 100 | 100 | 100 | 100 | 100 | 100 | 100 | 100 |
| (A) | | | | | | | | | |
| Component | TMPT | 44 | 52 | 52 | | | | | |
| (B) | A-TMPT | | | | | | | | 35 |
| | SR502 | 9 | 9 | 9 | 9 | 35 | 9 | 9 | 9 |
| | M313 | 23 | 23 | 23 | 23 | 23 | 23 | 23 | 23 |
| | (Trifunctional) | | | | | | | | |
| Other | M313 | 12 | 12 | 12 | 12 | 12 | 12 | 12 | 12 |
| monomers | (Bifunctional) | | | | | | | | |
| | DCP-A | | | | 35 | | | | |
| | A-DPH | | | | | | | | |
| | IB-X | | | | | | | | |
| | IB-XA | 17 | 17 | | 17 | 17 | 44 | 52 | 17 |
| Component | TPO | 2 | 2 | 2 | 2 | 2 | 2 | 2 | 2 |
| (C) | 84 | 8 | 8 | 8 | 8 | 8 | 8 | 8 | 8 |
| | 127 | | | | | | | | |
| | 55 | | | | | | | | |
| | 1173 | | | | | | | | |
| Component | DOS | 5 | 5 | 5 | 5 | 5 | 5 | 5 | 5 |
| (D) | | | | | | | | | |
| Total | | 220 | 228 | 211 | 211 | 202 | 203 | 211 | 211 |
| Total of the component (B) | | 78 | 84 | 84 | 32 | 58 | 32 | 32 | 67 |
| TMPT content ratio (%) | | 58 | 62 | 62 | 0 | 0 | 0 | 0 | 0 |
| relative to total of the | | | | | | | | | |
| component (B) | | | | | | | | | |
| Total of the component (C) | | 10 | 10 | 10 | 10 | 10 | 10 | 10 | 10 |
| TPO content ratio (%) | | 20 | 20 | 20 | 20 | 20 | 20 | 20 | 20 |
| relative to total of the | | | | | | | | | |
| component (C) | | | | | | | | | |
| | | Com- | Com- | Com- | Com- | Com- | Com- | Com- | |
| | | parative | parative | parative | parative | parative | parative | parative | |
| | Raw | Example | Example | Example | Example | Example | Example | Example | Comparative |
| Component | materials | 6 | 7 | 8 | 9 | 10 | 11 | 12 | Example 13 |
| Component | UF-8001G | 100 | 100 | 100 | 100 | 100 | 100 | 100 | 100 |
| (A) | | | | | | | | | |
| Component | TMPT | 5 | 19 | 9 | 19 | 19 | 19 | 19 | 35 |
| (B) | A-TMPT | | | | | | | | |
| | SR502 | 9 | 9 | 9 | 9 | 9 | | 9 | 9 |
| | M313 | | 23 | 23 | | 23 | 23 | 23 | |
| | (Trifunctional) | | 12 | 12 | | 12 | 12 | 12 | |
| Other | M313 | | | | | | | | |
| monomers | (Bifunctional) | | | | | | | | |
| | DCP-A | | | | | | | | |
| | A-DPH | | | | 35 | | | | |
| | IB-X | 35 | | | | | | 52 | |
| | IB-XA | | 35 | 44 | 35 | 35 | 35 | | 35 |
| Component | TPO | 4 | 5 | 2 | 2 | 2 | 2 | 2 | 2 |
| (C) | 84 | 8 | 8 | 8 | 8 | 8 | 8 | 8 | 8 |
| | 127 | | | | | | | | |
| | 55 | | | | | | | | |
| | 1173 | | | | | | | | |
| Component | DOS | 5 | 5 | 5 | 5 | 5 | 5 | 5 | 5 |
| (D) | | | | | | | | | |

| | | | | | | | | |
|---|---|---|---|---|---|---|---|---|
| Total | 166 | 216 | 212 | 213 | 213 | 204 | 230 | 194 |
| Total of the component (B) | 14 | 51 | 41 | 28 | 51 | 42 | 51 | 44 |
| TMPT content ratio (%) | 36 | 37 | 22 | 68 | 37 | 45 | 37 | 80 |
| relative to total of the | | | | | | | | |
| component (B) | | | | | | | | |
| Total of the component (C) | 12 | 13 | 10 | 10 | 10 | 10 | 10 | 10 |
| TPO content ratio (%) | 33 | 38 | 20 | 20 | 20 | 20 | 20 | 20 |
| relative to total of the | | | | | | | | |
| component (C) | | | | | | | | |

For the compositions produced, in Examples 1 to 12 and Comparative Examples 1 to 13, and cured products obtained by curing of the compositions, viscosity and heat generation upon curing of the composition, transmittance, outer appearance and glossiness after surface treatment of the cured products were evaluated according to the following method. The results are shown in Table 2.

### Measurement of Viscosity of Composition

The composition was collected in an amount of 0.5 ml, taken in a cup for measurement, and subjected to viscosity measurement at the following conditions with use of an EHD type viscometer (manufactured by TOKI SANGYO CO., LTD.). The results are given as “viscosity (Pa·s)”. From the viewpoint of the workability, the viscosity of the composition is preferably 30 Pa·s or less.

### Measurement Conditions

Cone rotor: 3°×R14

Rotation race: 10 rpm

Measurement time: 3 minutes

Measurement temperature; 25° C. (temperature is controlled by using an incubator).

### Measurement of Transmittance of Cured Product

The composition was flowed into a space between two pieces of a glass plate having an 800 μm metal spacer disposed therebetween such that the thickness of a cured product to be obtained is 800 μm. By using an ultraviolet ray illuminator of belt conveyer type which is provided with a high-pressure mercury lamp, a cured product was produced by illumination with cumulative light amount of 30 kJ/m². Based on visible-ultraviolet spectrophotometry, measurement was made for “transmittance (%)” as a percentage value of light amount before and after light transmission through a cured product at a wavelength of 450 nm, 410 nm, and 400 nm. In order for the cured product to exhibit a colorless and transparent outer appearance with no yellowing, the transmittance at a wavelength of 400 nm is preferably 10% or higher.

### Determination of Outer Appearance of Cured Product

The cured, product which has been produced for measuring the transmittance above was placed on a white paper, and by determining the presence or absence of coloration with a naked eye according to the following evaluation criteria, “outer appearance of cured product” is obtained. From the viewpoint that the cured product is required to be colorless when the composition is used for a topcoat, the result is preferably “O”.

### Evaluation Criteria

O: There is no coloration

x: There is coloration

### Determination of Glossiness After Surface Treatment of Cured Product

On top of a test piece which has been obtained by electrodeposition after blackening treatment of SPCC-SD (for the details, see JIS G 3141 (2011)), the composition prepared in the above was applied such that it has a thickness of 100 μm in dry state. After that, the composition was cured by irradiation for 10 seconds with an LED lamp for nails (standard voltage; 240 V, 50 to 60 Hz, power consumption; 30 W, and wavelength; 400 to 410 nm). After that, the surface of the obtained, cured product was wiped 3 times with cotton impregnated with ethanol. Ethanol was removed by drying, and the result of glossiness, which has been determined with a naked eye based on the following evaluation criteria, was obtained as “glossiness after surface treatment.” From the viewpoint of the outer appearance, the result is preferably “O” when the composition is used for a topcoat.

### Evaluation Criteria

O: There is glossiness

x: There is no glossiness

### Determination of Heat Generation Upon Curing of the Composition

The composition was applied on the nails of a human hand to have a thickness of 100 μm in a dry state and was cured by irradiation for 10 seconds with the LED lamp for nails. At the time of curing, “heat generation upon curing” was determined according to the following evaluation criteria. From the viewpoint of the workability, the result is preferably “O”.

### Evaluation Criteria

O: There is no hot feeling on nails

x: There is hot feeling on nails

### Measurement of Hardness of Cured Product

The composition was applied to have a thickness of 1 mm in a dry state, and by performing the light irradiation two times with cumulative light amount of 30 kJ/m² using an illuminator of belt conveyer type which is provided with a high-pressure mercury lamp, a sheet-like cured product was produced. When the cured product reaches 25° C., the cured product was overlaid to have three layers, and the resultant was used as a sample. While maintaining the pressurizing surface of a D type durometer (that is, hardness tester) to be parallel to the sample, the pressurizing surface and the sample were tightly adhered to each other by quickly applying force of 10 N without being accompanied by any impact. The maximum value was read at the time of measurement, and the maximum value was taken as “hardness (no unit)”. Details are based on JIS K 6253-3 (2012). When the hardness of a sample is D50 or higher, it can be said that the sample has hardness enough to be used in the application for a topcoat.

TABLE 2

| | | Example | Example | Example | Example | Example | Example | Example | Example | Example |
|---|---|---|---|---|---|---|---|---|---|---|
| Test item | | 1 | 2 | 3 | 4 | 5 | 6 | 7 | 8 | 9 |
| Viscosity | | 17 | 20 | 16 | 16 | 16 | 16 | 16 | 9 | 22 |
| Transmittance | 450 nm | 98 | 98 | 98 | 92 | 95 | 96 | 99 | 97 | 98 |
| | 410 nm | 62 | 62 | 63 | 57 | 60 | 64 | 68 | 65 | 63 |
| | 400 nm | 16 | 18 | 17 | 13 | 15 | 16 | 19 | 23 | 19 |

| | | | | | | | | | |
|---|---|---|---|---|---|---|---|---|---|
| Outer appearance of | ◯ | ◯ | ◯ | ◯ | ◯ | ◯ | ◯ | ◯ | ◯ |
| cured product | | | | | | | | | |
| Glossiness after surface | ◯ | ◯ | ◯ | ◯ | ◯ | ◯ | ◯ | ◯ | ◯ |
| treatment | | | | | | | | | |
| Heat generation upon | ◯ | ◯ | ◯ | ◯ | ◯ | ◯ | ◯ | ◯ | ◯ |
| curing | | | | | | | | | |
| Hardness | D64 | D60 | D65 | D63 | D63 | D64 | D63 | D65 | D75 |

| | | | | | Com- | Com- | Com- | Com- | |
|---|---|---|---|---|---|---|---|---|---|
| | | | | | parative | parative | parative | parative | |
| | | Example | Example | Example | Example | Example | Example | Example | Comparative |
| Test item | | 10 | 11 | 12 | 1 | 2 | 3 | 4 | Example 5 |
| Viscosity | | 15 | 13 | 26 | 18 | 14 | 14 | 10 | 17 |
| Transmittance | 450 nm | 98 | 98 | 98 | 97 | 97 | 99 | 98 | 97 |
| | 410 nm | 62 | 62 | 62 | 59 | 59 | 59 | 61 | 60 |
| | 400 nm | 17 | 18 | 18 | 16 | 16 | 14 | 16 | 18 |
| Outer appearance of | | ◯ | ◯ | ◯ | ◯ | ◯ | ◯ | ◯ | ◯ |
| cured product | | | | | | | | | |
| Glossiness after surface | | ◯ | ◯ | ◯ | X | X | X | X | X |
| treatment | | | | | | | | | |
| Heat generation upon | | ◯ | ◯ | ◯ | ◯ | ◯ | ◯ | ◯ | ◯ |
| curing | | | | | | | | | |
| Hardness | | D66 | D67 | D63 | D65 | D60 | D63 | D65 | D66 |
| | | Com- | Com- | Com- | Com- | Com- | Com- | Com- | |
| | | parative | parative | parative | parative | parative | parative | parative | |
| | | Example | Example | Example | Example | Example | Example | Example | Comparative |
| Test item | | 6 | 7 | 8 | 9 | 10 | 11 | 12 | Example 13 |
| Viscosity | | 25 | 16 | 13 | 22 | 15 | 17 | 7 | 16 |
| Transmittance | 450 nm | 98 | 98 | 98 | 98 | 99 | 98 | 98 | 96 |
| | 410 nm | 31 | 38 | 62 | 62 | 62 | 62 | 60 | 61 |
| | 400 nm | 1 | 2 | 17 | 17 | 16 | 17 | 16 | 17 |

| | | | | | | | | |
|---|---|---|---|---|---|---|---|---|
| Outer appearance of | X | X | ◯ | ◯ | ◯ | ◯ | ◯ | ◯ |
| cured product | | | | | | | | |
| Glossiness after surface | X | X | X | X | X | X | X | X |
| treatment | | | | | | | | |
| Heat generation upon | ◯ | ◯ | ◯ | X | ◯ | ◯ | ◯ | ◯ |
| curing | | | | | | | | |
| Hardness | D65 | D69 | D65 | D74 | D65 | D70 | D75 | D70 |

When comparisons are made between Examples 1 to 12 and Comparative Examples 1, 3, 4, 6, 8, 11, and 13, it was found that, in Comparative Examples in which the content of the component (B) is out of the range of 50 to 100 parts by mass relative to 100 parts by mass of the component (A), no glossiness was exhibited on a surface of the cured product after wiping. Furthermore, when comparisons are made between Examples 1 to 12 and Comparative Examples 2, 5, 7, 10, and 12, it was found that, in Comparative Examples in which the content, of the component (B) is in the range of 50 to 100 parts by mass relative to 100 parts by mass of the component (A) but the content of trimethylol propane trimethacrylate is out of the range of 40 to 70% by mass relative to the total of the component (B), no glossiness was exhibited on a surface of the cured product similarly to the above. Furthermore, in Comparative Example 9 in which part of the component (B) is replaced with a hexafunctional (meth)acrylic monomer, there is heat generation upon curing, and therefore it is not acceptable in terms of practical application.

Furthermore, according to Comparative Examples 6 and 7, the visible light type photopolymerization initiator is more than 30% by mass relative to the total amount of the component (C), and thus a transparent outer appearance of a cured product was not obtained. It is considered chat this tendency corresponds to the result that the transmittance of a cured product with a thickness of 800 pa is lower than 10.0% at a wavelength of 400 nm.

### INDUSTRIAL APPLICABILITY

The present invention relates to a photocurable composition, which is suitable for a topcoat of nails or artificial nails and can exhibit excellent glossiness and a transparent and colorless outer appearance, even after performing an operation of wiping out a non-cured part in a topcoat having oxygen inhibition with a solvent which follows the practices in the field of nails, and thus the photocurable composition can be widely used in the field of nails.

The application of the present invention is not limited to the embodiments described above, and suitable modifications can be made within a range in which the spirit of the present invention is not impaired.

The present application is based on Japanese Patent Application No. 2014-224794 filed on Nov. 5, 2014, and its enclosure is entirely incorporated herein by reference.

1. A photocurable composition for a topcoat of nails or artificial nails comprising the following components (A) to (C), comprising 40 to 70% by mass of trimethylol propane trimethacrylate relative to the total amount of the component (B), and satisfying that a cured product thereof with a thickness of 800 mm has transmittance of 10.0% or higher at a wavelength of 400 nm:
component (A): a (meth)acrylic oligomer;
component (B): a trifunctional (meth)acrylic monomer contained at 50 to 100 parts by mass relative to 100 parts by mass of the component (A); and
component (C): a photopolymerization initiator contained at 1 to 20 parts by mass relative to 100 parts by mass of the component (A).

2. A photocurable composition for a topcoat of nails or artificial nails comprising the following components (A) to (C), comprising 40 to 70% by mass of trimethylol propane trimethacrylate relative to the total amount of the component (B), and comprising 0 to 30% by mass of a visible light type photopolymerization initiator relative to the total amount of the component (C):
component (A): a (meth)acrylic oligomer;
component (B): a trifunctional (meth)acrylic monomer contained at 50 to 100 parts by mass relative to 100 parts by mass of the component (A); and
component (C): a photopolymerization initiator contained at 1 to 20 parts by mass relative to 100 parts by mass of the component (A).

3. The photocurable composition for a topcoat of nails or artificial nails according to claim 2, wherein a cured product thereof with a thickness of 800 mm has transmittance of 10.0% or higher at a wavelength of 400 nm.

4. The photocurable composition for a topcoat of nails or artificial nails according to claim 2, further comprising a plasticizing agent as the component (D).

5. A coating film obtained by curing the photocurable composition for a topcoat of nails or artificial nails according to claim 2 by light irradiation and then wiping out a surface of the cured product with a piece of cloth impregnated with a solvent.

6. A method for exhibiting glossiness by curing the photocurable composition for a topcoat of nails or artificial nails according to claim 2 by light irradiation and then wiping out a surface of the cured product with a piece of cloth impregnated with a solvent.
